(19) United States
(12) Patent Application Publication
(10) Pub. No.: US 2021/0345942 A1
(43) Pub. Date: Nov. 11, 2021

(54) Method and Device for Determining Nature or Extent of Skin Disorder

(71) Applicant: Veronica Kinsler, London (GB)

(72) Inventors: Veronica KINSLER, London (GB); Stuart ROBSON, London (GB); Jan BOEHM, London (GB)

(21) Appl. No.: 17/282,644

(22) PCT Filed: Sep. 30, 2019
(86) PCT No.: PCT/GB2019/052753

§ 371 Date: Apr. 2, 2021

(30) Foreign Application Priority Data
Oct. 2, 2018 (GB) 1816083.8

(51) Int. Cl. A61B 5/00; G06K 9/32; G06T 7/00; G06K 9/62; G06T 7/62; G06T 7/70; G06T 7/50; G16H 50/20; G16H 30/40

(52) U.S. Cl. CPC A61B 5/441; A61B 5/0077; A61B 5/7267; G06K 9/3233; G06T 7/0012; G06K 9/6267; G06K 2209/05; G06T 7/70; G06T 7/50; G16H 50/20; G16H 30/40; A61B 2576/02; G06T 2207/30088; G06T 7/62

(57) ABSTRACT

A system and computer-implemented method for quantifying the severity of a skin disorder. The method comprises: receiving one or more images of skin of a subject; determining a surface model of the skin of the subject; mapping the one or more images of the skin of the subject onto the surface model to form an image-mapped surface model of the skin of the subject; and determining a value for the severity of a skin disorder based on the image-mapped surface model of the skin of the subject.

### TECHNICAL FIELD

The present disclosure relates to methods and devices for quantifying the nature and/or extent of a skin disorder.

### BACKGROUND

Skin diseases constitute the 10th greatest cause of disability-adjusted life years in the UK, defined as the number of years lost due to ill-health, disability or early death (Newton, J. N. et al. Changes in health in England, with analysis by English regions and areas of deprivation, 1990-2013: a systematic analysis for the Global Burden of Disease Study 2013. Lancet 386, 2257-74 (2015)). This results in heavy socioeconomic costs, with a total cost to the NHS in England for 2010/2011 of £2,135,000,000 (excluding the substantial costs of burn injuries), and $75 billion of direct costs in the USA in 2013 (Lim, H. W. et al. The burden of skin disease in the United States. J Am Acad Dermatol 76, 958-972 e2 (2017)). In addition, these costs are rising steeply, due to new treatment modalities and aging populations. Optimisation of management of skin disease is therefore a high priority.

A key factor in clinical management of any disease is accurate and objective measurement of severity. This does not currently exist for skin disease. The reason for this is that it has been technically difficult. Most skin diseases are “multi-focal”, affecting various parts of the body. In addition, the skin is the only organ system that cannot be measured by radiological methods such as X-ray or ultrasound.

Existing measurement methods for skin are semi-quantitative, inaccurate, and operator-dependent (Schmitt, J., Langan, S., Williams, H. C. & European Dermato-Epidemiology, N. What are the best outcome measurements for atopic eczema? A systematic review. J Allergy Clin Immunol 120, 1389-98 (2007)). These measurement methods may use a tape measure or the rule of nines (burns, vitiligo), visual estimation of affected area (psoriasis, birthmarks), or severity scores such as Patient Oriented Eczema Measure (eczema). These are inherently subjective and inaccurate. In addition, these methods are not generally used outside specialist centres.

Existing imaging systems for skin such as mole mapping are unable to measure the total extent/severity of a skin disease. They are limited in that they are only imaging systems for monitoring change of individual areas, and are not able to provide a total skin measurement of extent and severity across the entirety of the body.

The lack of accurate and objective measurement for whole body skin disease has a serious impact on patients, as well as doctors and hospitals. There is no accurate baseline measurement of severity, and therefore no consistent stratification of patients for treatment. Patients are usually treated sequentially by a treatment ladder when treatments fail, with no accurate numeric documentation of response or lack of response. Not only does this likely result in less effective treatment, but also likely incurs associated socioeconomic costs both to the patient and the NHS, in the form of increased number of appointments in secondary and tertiary care, and loss of school and work time.

Furthermore, without accurate measurement of extent or severity of skin disease there are no clear guidelines on the amounts of topical therapy to prescribe. This can result in doctors prescribing an incorrect dosage. Underestimating the required dosage can result in a reduced effectiveness of the treatment. Overestimating the required dosage can result in substantial wastage. Prescriptions for topical skin therapies are a major cost, and an inability to measure the amount required by a patient will inevitably lead to inaccurate prescription. Globally this problem is exacerbated even further, with a total lack of skin disease severity measurement in most parts of the world.

There is therefore a need for a means for accurately quantifying the nature and severity of skin diseases.

### SUMMARY

According to a first aspect there is provided a computer-implemented method for quantifying the nature and/or extent of a skin disorder. The method comprises: receiving one or more images of skin of a subject; determining a surface model of the skin of the subject; mapping the one or more images of the skin of the subject onto the surface model to form an image-mapped surface model of the skin of the subject; and determining a value for the nature and/or extent of a skin disorder based on the image-mapped surface model of the skin of the subject.

The implementations described herein provide an accurate and objective quantification of the nature and/or extent of a skin disorder. The skin disorder may be any form of skin condition that can be detected via changes in the spectral properties of the affected regions. By mapping images onto a surface model the severity of the skin disorder can be determined even where the skin disorder is multi-modal or wraps around body parts. This can be achieved by receiving images taken of the subject from a variety of angles around the subject (in the round). This allows the images to be mapped onto the model effectively to allow effective quantification of the nature and/or extent of the skin disorder.

The nature and/or extent can be used to determine the overall severity of the skin disorder. The severity can be quantified based on the extent (e.g. the surface area) affected, or the acuteness of the affected area(s) (e.g. the severity per unit area or the intensity/acuteness of the condition within any affected areas). The severity may be measured based on the spectral properties of the affected areas.

Images of skin need not be taken by the system at the time of quantification. Instead, the images could be taken of the subject in advance and accessed from a database of images (e.g. stored locally in memory or accessed via a network).

According to a further implementation the method further comprises identifying one or more regions of skin that are affected by a skin disorder based on the image-mapped surface model of the skin, and the value for the nature and/or extent of the skin disorder is determined based on the one or more regions of skin that are affected by the skin disorder. Accordingly, the method may be able to classify areas of the model as affected by the skin disorder.

According to a further implementation the method further comprises receiving a set of characteristics of skin that is affected by the skin disorder, wherein the identification of the one or more regions of skin that are affected by a skin disorder is based on the set of characteristics.

The characteristics could be a difference in colour and/or shading of skin relative to a reference area of skin. The reference area may be an area not affected by the skin disorder (e.g. healthy skin) or an area of reduced severity of the skin disorder. The characteristics could be received by accessing characteristics stored by the computer system or receiving characteristics input from an external system or by a user.

Alternatively or in addition, the characteristics could be received through analysis of a section of the image mapped surface model (or of one of the images) that has been identified as being affected by a skin condition and/or analysis of a section of the image mapped surface model (or of one of the image) that has been identified as showing the reference area.

According to a further implementation the one or more regions of skin that are affected by a skin disorder are identified using a machine learning classifier. The machine learning classifier may be pre-trained based on pre-classified training data.

According to a further implementation determining the value for the nature and/or extent of the skin disorder comprises one or more of: determining the surface area of the one or more regions of skin that are affected by the skin disorder; determining the proportion of the overall surface area of skin that is affected by the skin disorder; determining one or more of an average discolouration, an average shading, or an average colouring of the one or more regions of skin that are affected by the skin disorder; and determining, within the one or more regions of skin that are affected by the skin disorder, the strength of one or more spectral signatures associated one or more corresponding molecules that characterise the skin disorder.

Accordingly, the nature, extent and/or severity may quantified based on the area of skin affected and/or the spectral properties of the affected area(s). The colour and/or shading may be as compared to the colour and/or shading of skin not affected by the skin disorder (e.g. to obtain discolouration or a change in shading). The severity value may be proportional to the difference between the colour and/or shading of the area affected by the skin disorder and the colour and/or shading of the area not affected by the skin disorder.

Furthermore, where a particular spectral signature is known to be associated with one or more molecules that are indicative of the skin disorder, the spectral signature may be detected and measured in order to identify the skin disorder and determine the value for the nature, extent and/or severity of the skin disorder. A spectral signature may be a particular frequency or set of frequencies associated with the one or more molecules. Where the spectral signature includes a particular set of frequencies, the signature may also define a particular range of ratios of intensity/strength for the relevant frequencies. The strength of the spectral signature may be determined based on the intensity (e.g. the average intensity) of the signature within the one or more regions of skin that are affected by the skin disorder.

According to a further implementation the method further comprises identifying one or more regions of skin not affected by the skin disorder. This allows the characteristics of the affected regions to be compared to the unaffected regions (e.g. healthy skin) to more accurately measure the severity of the skin condition.

According to a further implementation, the method further comprises receiving a further set of characteristics, the further set of characteristics being of skin that is not affected by the skin disorder, and the identification of the one or more regions of skin not affected by the skin condition is based on the further set of characteristics.

The further set of characteristics may be received in a similar manner to the characteristics for skin affected by a skin disorder (e.g. stored, input, or determined from an area of the image-mapped surface model identified by the user to not be affected by the skin disorder).

According to a further implementation the method comprises receiving position data indicating the shape of the skin of the patient and wherein the surface model is determined based on the position data. This allows the surface model (and therefore the severity value) to be more accurately and more efficiently determined. The position data could be range data, e.g. range data obtained via active range measurements (e.g. from a 3D camera). The position data need not be measured as part of the method, but could instead be accessed from a database of measurements (e.g. stored locally or accessed via a network).

According to a further implementation the surface model is determined based on the one or more images. These images may be taken from various positions around the subject. The boundaries of the object may be determined using object recognition methods. The determination of the surface model based on the images could be in addition to determining the model based on position data (i.e. in combination with), or could be without the use of position data (i.e. from the images alone).

According to a further implementation the method further comprises correcting colour and/or shading in the image mapped surface model to account for surface geometry, lighting or camera angles.

According to a further aspect there is provided a system for quantifying the severity of a skin disorder, the system comprising a processor configured to implement any method described herein.

Images of skin need not be taken by the system, but could instead be accessed from a database of images (e.g. locally stored or via a network).

According to a further implementation there is provided a non-transitory computer readable medium comprising computer executable instructions that, when executed by a computer, cause the computer to implement any method described herein.

### BRIEF DESCRIPTION OF THE DRAWINGS

Arrangements of the present invention will be understood and appreciated more fully from the following detailed description, made by way of example only and taken in conjunction with drawings in which:

FIG. 1 shows a system for determining the nature and/or extent of a skin disease according to an embodiment of the invention;

FIG. 2 shows a method for quantifying the nature and/or extent of a skin disease according to an embodiment

FIG. 3 shows a method of calibrating a system according to the embodiments described herein;

FIG. 4 shows a computing system for determining the nature and/or extent of a skin disorder according to an embodiment; and

FIG. 5 shows classified images and a classified surface model produced according to an embodiment for a single subject.

### DETAILED DESCRIPTION

The embodiments described herein provide a total skin disease measurement system. This provides a revolutionary step beyond existing imaging/monitoring systems for skin. The embodiments described herein obtain accurate and objective numeric measurements of total surface area affected by any skin condition (skin disease, or any other form of skin abnormality), and its characteristics, including for extensive and multifocal diseases. Importantly, this system can be implemented using images and measurements taken with off-the-shelf products. The methods can be applied either on-line or off-line to re-analyse and compare historical patient image data with current images. Accordingly, previously taken 2D and 3D images may be analysed off-line to determine the extent of the skin condition.

Improvement in the management of skin diseases is a key priority for health services. There is a steeply increasing budget for topical and systemic therapies within the UK and US over the last decade. Similarly, this is a priority for patients, as skin diseases such as eczema have become increasingly common, now affecting one third of children. Associated patient costs are in quality of life for the patient and families/carers, loss of school and work time, and high costs of travel to secondary and tertiary care centres.

Multifocal diseases are diseases that affect two or more locations on the body. Given that the skin is the largest organ of the body, and has a large surface area, these multiple locations may be spaced apart from each other, on different body parts, or located on opposite sides of the body. Furthermore, given the geometry of the body, skin diseases can be difficult to measure as they can wrap around one or more sections of the body. This makes it difficult to image and quantify skin diseases, and in particular, multi-focal skin diseases.

Current methods are unable to accurately and objectively measure these types of diseases. The methods described herein are applicable to measuring a variety of skin diseases including: a) Psoriasis, b) congenital melanocytic naevi (CMN), c) vitiligo, d) eczema, and e) Inflammatory Linear Verrucous Epidermal Nevus (ILVEN).

Furthermore, it can be difficult to quantify the extent or severity of skin conditions accurately across different subjects as the appearance skin conditions depends on the skin type of the individual (e.g. skin colour). For instance, it is often difficult to detect some skin diseases on darker skin by eye, particularly in fine detail. In addition, as skin type can vary significantly between subjects, this makes it difficult to provide an objective measure of skin conditions across different subjects. The implementations described herein overcome this problem by classifying areas affected by a skin condition based on the difference in colour and texture relative to one or more areas of healthy skin on the subject. Furthermore, the implementations make use of one or more cameras that provide improved colour characterisation relative to the naked eye.

Geometric and radiometric calibration of the imaging system allow different wavelengths and colour channels to be selected in order to maximise the optical signal of a given disease vs healthy skin. For instance, the intensity of red on black skin is a useful quantifier of the severity of eczema.

Given the large surface area and multi-focal nature of many skin diseases, they are impossible currently to quantify as a total affected surface area, with respect to total body surface area. In addition, the severity and nature is difficult to quantify accurately. Most diseases do not have measurement system for severity, whereas common diseases such as eczema have multiple competing severity systems. In addition, the nature and severity of skin diseases at different ages can be difficult to characterise due to the large variation in body size and shape between subjects, and in particular due to differential growth of different body parts at different ages. Furthermore, the large variety of skin colours means that accurate identification and characterisation of affected areas can be difficult.

The embodiments described herein introduce the Skin Sizer. This provides a radical innovation in skin disease management. Anticipated outcomes will vary with the skin condition. In general, the anticipated benefits include:
1) Reduction in time to instigation of appropriate patient treatment through accurate and objective patient stratification, leading to decreased secondary/tertiary care outpatient appointments and GP appointments, and to decreased drug costs;
2) Reduction in number of unnecessary investigations through better patient stratification;
3) Reduction in clinician time during appointments, with measurement performed before seeing the doctor (3-5 minutes) and eliminating the need for existing classification and severity scores (5-10 minutes);
4) Reduced prescription wastage of topical therapies by accurate and objective measurement of surface area of affected skin at each visit (and prescribing of the correct volumes of creams);
5) Increased quality of life of patients and families due to all of the above;
6) Reduced socioeconomic burden on the UK economy from loss of school and work attendance, and costs of travel to secondary/tertiary centres;

There is overwhelming evidence from the literature from other diseases that accurate, objective disease severity measurement improves patient care and ultimately reduces costs. Some key severity measurements in recent medical history include left ventricular ejection fraction in cardiovascular disease, and glomerular filtration rate in renal disease, both of which rapidly became key severity measures for stratification of patient outcome and disease management.

As the first real measurement system for multifocal skin disease, the embodiments described herein are able to provide the same order of magnitude impact on skin disease management as the measurement of left ventricular ejection fraction and glomerular filtration rate in their relevant medical fields.

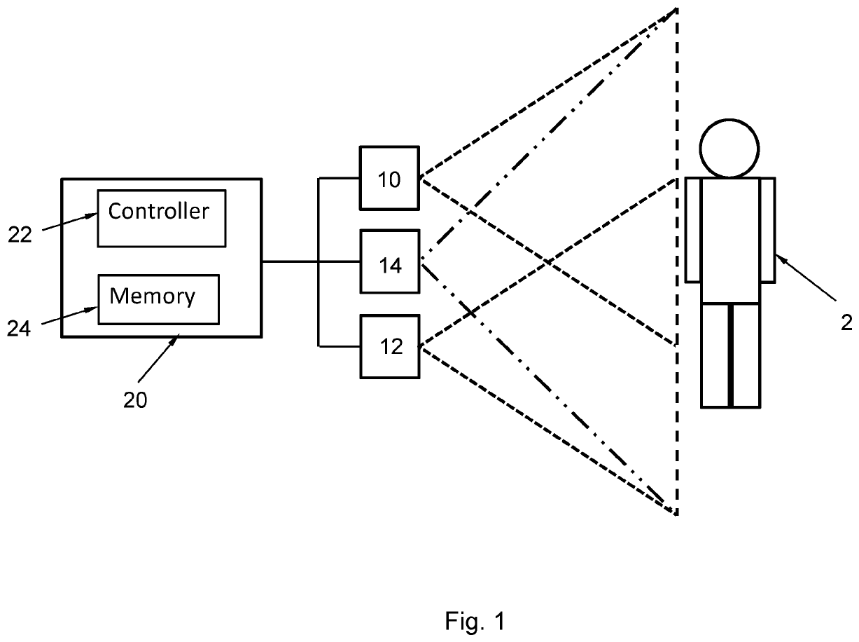

FIG. 1 shows a system for determining the nature and/or extent of a skin disease according to an embodiment of the invention. The system comprises two cameras 10, 12 for recording the colour of the skin of a subject 2 (e.g. a patient) and a range camera 14 for recording range measurements so that a 3D surface model of the subject can be generated.

The cameras 10, 12 can be called two dimensional (2D) cameras as they output 2D images of the colour of the skin of the subject 2. Each camera 10, 12 outputs data indicating the colour detected by the camera at each pixel. Each of the 2D cameras 10, 12 may be any generally available digital camera, such as a DSLR or a monochrome camera equipped with one or more coloured filters tuned to the skin tone and disease being investigated. In each case the imaging geometry and spectral response on the imaging system can be quantified in order to deliver accurate, precise and reliable mapping between information captured at each pixel and the total skin surface of the patient

To improve the accuracy of the colour readings and applicability to a wide range of imaging situations, the images taken by the cameras may be lit using electronic flashes or controlled photographic studio lighting. A colour chart can also be located within the field of view of each camera to allow the spectral properties of the environment to be characterised. This provides a consistent calibration and colour correction for improving the accuracy of the images. Preferably the colour chart is located adjacent to the subject 2, in the plane of sharp focus.

The range camera 14 can be called a three dimensional (3D) camera as it outputs range data of the skin of the subject 2 that can be used to determine a surface model of the skin. The range camera 14 outputs data in the form of a 2D image indicating the distance between the sensor and the subject at each pixel. The range camera 14 may be any generally available range camera, one such example is the ASUS™ XTION 3D range camera.

The cameras 10, 12 and range camera 14 output their respective images to a computing system 20. The computing system 20 is configured to combine the images to produce photogrammetric colour corrected images of the skin of the subject 2 for determining the nature and/or extent of a skin disease for the patient. The measurements by the cameras 10, 12 and range camera 14 are taken simultaneously to ensure that the measurements can be combined to produce an accurate 3D representation of the skin independent of patient body motion.

The relative positions and orientations of the cameras 10, 12 and the range camera 14 are known through a photogrammetric calibration procedure. The cameras 10, 12 and range camera 14 are mounted on a rigid, but mobile structure. This ensures that, after the system has been calibrated to determine the relative positions of the sensors, the images from the cameras 10, 12 may be combined with the range measurements by the computing system 20 to obtain a 3D surface model of the skin of the subject.

The field of view of the two cameras 10, 12 overlaps the field of view of the active camera 14 across the entirety of an imaging region that contains the subject 2. In the present embodiment, the field of view of each camera 10, 12 overlaps the field of view of the other by around 50%. The field of view of the range camera 14 overlaps the fields of view of both of the cameras 10, 12.

In order to get full coverage of the skin of the subject 2, it may be necessary to take measurements from multiple viewpoints relative to the subject 2. This can be achieved by turning the subject relative to the cameras 10, 12, 14. Alternatively, multiple sets of cameras 10, 12 and range cameras 14 may be arranged around the subject to get full 360° coverage. This can ensure that measurements covering all angles can be taken simultaneously, to improve the accuracy of the 3D model once the measurements have been combined.

In one embodiment, the cameras 10, 12 and range camera 14 are capable of capturing image sequences or videos. In this embodiment, the cameras 10, 12 and range camera may take a stream of measurements synchronised in time such that the required measurements can be obtained by turning the subject 2 in the field of view of the cameras whilst the image sequences are being recorded. Time-synchronised measurements can then be output for analysis by the computing system 20. These time-synchronised measurements may be all of the measurements taken over time or a subset (a sample) of the measurements taken over time.

The present embodiment employs two cameras 10, 12 and a single range camera 14 arranged vertically (e.g. each located along a single vertical axis) to ensure that there is sufficient coverage across the entire height of the subject 2 at the required resolution for accurate measurement of skin diseases. Nevertheless, alternative embodiments can employ differing numbers of cameras based to make use of varying properties of differing cameras and range cameras.

For instance, a single camera and a single range camera may be utilised, provided that the field of view of the two cameras overlaps to form an imaging volume. This may be achieved with coverage sufficient to cover the height of the subject 2 if a camera with a sufficiently high resolution is utilised at a sufficient distance from the subject 2 (or with an appropriate wide-angle lens). Alternatively, a single camera may be utilised with only partial imaging coverage across the height of the subject. This would then require multiple sets of images to be taken at differing heights in order to fully map the skin of the subject 2.

The embodiment of FIG. 1 utilises RGB cameras configured to detect light in the optical wavelength range. Alternative embodiments may make use of cameras operating over differing wavelengths, for instance, ultraviolet or infrared cameras. Alternatively, black and white cameras may be used. This can allow different spectral wavelengths to be analysed when quantifying the extent of skin conditions having different spectral properties.

Whilst the embodiment of FIG. 1 shows a computing system 20 connected directly to the cameras 10, 12 and range camera 14, the measurements of the subject do not need to be taken at the same time and at the same location as the analysis. In fact, alternative embodiments apply the methods described herein to historical image data stored in a database. It is therefore possible to analysis pre-existing images to obtain a severity measurement for the skin condition.

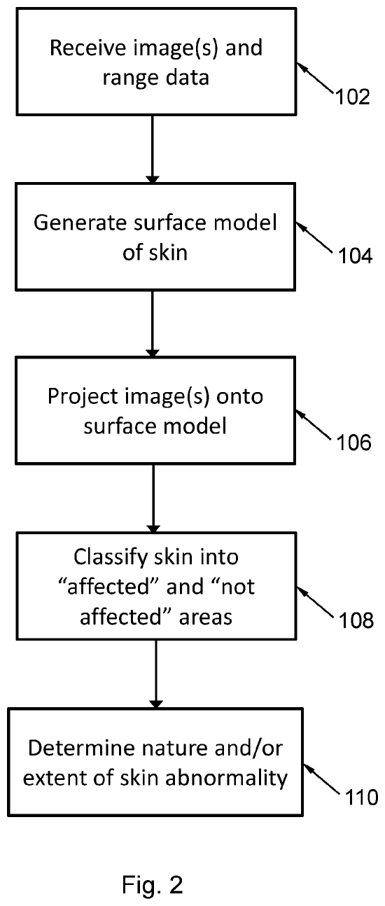

FIG. 2 shows a method for quantifying the nature and/or extent of a skin disease according to an embodiment. This method may be performed in the computing device 20 of FIG. 1.

Prior to measurement of a subject, the system must first be calibrated (not shown). Baseline calibration measurements can be taken to ensure that the colour images necessary for colour classification are geometrically registered with the range data from the 3D camera. The calibration method shall be discussed in more detail later.

Colour images and range data are received 102. Data obtained from all three cameras (the 2D cameras and the 3D camera) can be stored in memory of the computing system 20, along with patient meta-data and photogrammetric bundle adjustment logs for assessing precision and accuracy. Alternatively, the data may be stored in a database and the receiving step 102 may instead comprise accessing the data stored in the database.

A data fusion method then registers colour images and 3D range data to pixel map the high-resolution image data onto a dense triangle mesh. To achieve this, a surface model of the skin is determined from the range data 104. This can be achieved by combining range measurements taken from various angles around the subject. This produces a 3D model of the surface of the skin of the subject. The surface model may be divided into a polygon mesh, such as a triangle mesh. Then, the colour images are projected onto the 3D surface model 106. This may be achieved by projecting the colour data from each pixel back onto its corresponding polygon within the polygon mesh, or by projecting the polygons into the images and extracting the 2D pixel data within each polygon. In either case decisions are made utilising range camera surface normals compared to each colour camera, individual camera magnification of the local skin surface, local image sharpness, the presence of occlusions, local skin surface secularity, shadows and non-skin content such as clothes and hair. The result is an image-mapped surface representation of the body.

The image-mapped surface model is then analysed to classify the skin into one or more regions affected by a skin condition and one or more regions not affected by a skin condition 108. This may be achieved via a machine-learning classifier that is trained to detect one or more skin conditions. The user may select one or more skin conditions for classification, or the system may automatically detect the type of skin conditions based on the properties of the affected area(s).

The spectral absorbance of skin indicates the make up of that area of skin, which may then be disrupted by illness. The characteristics of healthy skin vary between subjects. For instance, melanin has a known spectral absorbance and variations in melanin are the main determinant in the variation in healthy skin colour between races.

Nevertheless, the characteristics of healthy skin on a specific patient can be used to determine whether any region of skin is affected by a skin condition. For instance, an area of reduced melanin (relative to the healthy skin of the subject) can indicate the presence of vitiligo (in which melanin is destroyed). In addition, haemoglobin also has a known spectral absorbance and a change in this absorbance can indicate a skin abnormality, for instance, via an increase in the redness of the skin. Collagen is a further component that has a characteristic spectral absorbance and that varies in quantity in various skin conditions.

The embodiments described herein make use of these known effects to classify areas of skin affected by a skin condition and to quantify the severity of the skin condition. The severity may be measured based on the extent of skin that is affected (the area of skin that is affected). The severity can also be measured based on the intensity (or acuteness) of the skin condition in the affected area(s). This can be indicated by the difference in spectral properties relative to healthy skin (or any other reference area of skin). For instance, the severity of a skin condition (such as eczema) can be indicated by the amount of increased haemoglobin (increased redness) indicated in the affected regions. Equally, a reduction in melanin can be indicative of an increased severity of vitiligo and can be indicated by a change in the spectral properties (e.g. red, yellow, brown and/or black colours) of the affected areas.

The difference in spectral properties may be measured relative to healthy skin or a different type of reference region. For instance, the reference region may be an area affected by other skin conditions but not affected by the skin condition being quantified.

Equally, the reference region may be a region of previously known abnormal skin in order to quantify the severity of new skin regions. Furthermore, the reference region may be a region of reduced severity compared to the region being quantified.

The method then determines the nature and/or extent of the skin disease based on the classified model 108. This may be determined by determining the amount of skin that is affected by the skin condition. For instance, the percentage of the overall surface area that is affected by the skin condition may be calculated. Alternatively, or in addition, the surface area of the affected area, such as in m², may be determined.

Alternatively or in addition, the colour of the affected area(s) can be analysed to determine the severity of the skin condition in the affected areas. As discussed, skin discolouration is indicative of a skin condition. The severity of the condition is indicated by the amount of discolouration. The average skin colour across the affected area(s) may be determined and compared to the average colour of the unaffected area(s). The average discolouration of the affected areas can then be used to quantify the severity of the condition.

The methods described herein provide an accurate, rapid and objective calculation of the nature, extent or severity of multifocal skin disease. Colour reflectance and surface normal data information can provide an accurate map of skin surface colour which allows the identification of diseased skin and calculation of total area of diseased skin as a percentage of total skin.

The method of FIG. 2 utilises a classifier for identifying regions that are affected by a skin condition and regions that are not affected by a skin condition. This classifier can be trained via machine learning in order to identify these regions. This training can involve classifying a set of labelled training data. The training data may be already classified. This may be input manually by a user in order to identify the affected regions. The classifier can then be trained on the training data, for instance, via reinforcement learning. The classifier may be trained to identify a specific abnormality, any form of abnormality, or to classify affected areas based on the abnormality so that the type of abnormality is identified.

To identify the affected region(s), the system receives an input from the user identifying one or more regions of abnormal skin (i.e. regions affected by the skin condition) as well as one or more reference regions of skin (e.g. regions not affected by the skin condition). This allows the system to take into account variations in skin colour when classifying the skin of the subject. The pre-trained classifier uses the spectral properties of the reference and abnormal regions to divide the surface model into abnormal regions and reference regions.

Prior to classification, the method may utilise a colour correction step. After the images are projected onto the surface model, the system may correct the colouring of the model. This may be based on the range data and/or the geometry of the model. For instance, the image content may be corrected based on surface geometry to account for oblique angles between camera and skin and fall off in illumination. This can help to make the colouring of the model more accurate and therefore increase the accuracy of the classification and quantification of the skin abnormality.

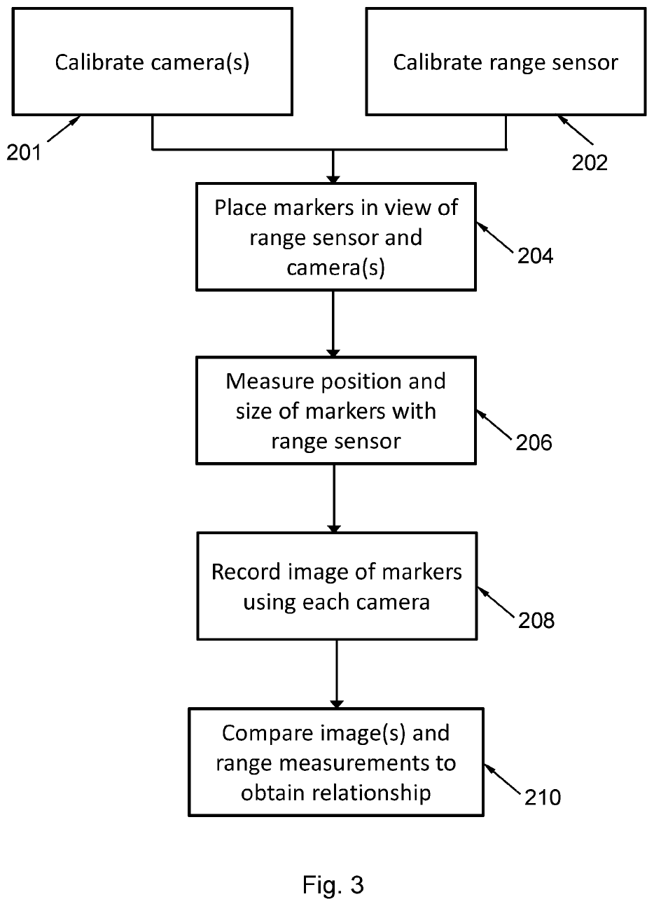

FIG. 3 shows a method of calibrating a system according to the embodiments described herein. This method may be applied to the embodiment of FIG. 1.

Firstly, the camera(s) 201 and range camera 202 are calibrated. The camera(s) are calibrated by assessing their spectral response to given wavelengths of light, for example from a mono-chromator or a set of narrow band filters with known transmission characteristics. Information from the colour chart recorded under the same lighting conditions as the patient can then be used to correct the colour of at each pixel in the image. Comparison between registered pixels from each of the cameras improves the capability of the system to make reliable measurements. This allows the system to account for any changes in the properties of the environmental lighting. The range camera is calibrated by taking range measurements of an object of known size. This allows the system to determine the scale of the range measurements.

Photogrammetric markers are placed within the field of view of the range camera and camera(s) 204. Then, the position and size of the markers are measured with the range camera 206. These are detailed in a range image output from the range camera 206. Without moving the markers, an image of the markers is taken using each camera 208. The camera image(s) and range measurements may be taken simultaneously.

The camera and range image are then compared to obtain a relationship between the pixels in the images and the range measurements. The coordinates of the markers within each image are determined. Equally, the coordinates of the markers within the range image are determined. The coordinates from the range data are interpolated at subpixel marker image coordinates. The coordinates are then used as control points for photogrammetric processing using a least squares bundle adjustment solution to simultaneously obtain best estimates of the imaging geometry of each camera and the geometric relationship between each camera and the photogrammetric markers.

The markers may be of a known size and shape. Accordingly, the markers can be used to calibrate the cameras and range sensor (steps 201 and 202) at the same time as registering the cameras with the range sensors (steps 206-210).

The geometric calibration between the camera and range images allows colour data from the images to be accurately registered with the range data. The photogrammetric calibration parameters and their variance covariance matrices output from the bundle adjustment describe both the internal and external camera imaging geometry and its uncertainty. This functional and stochastic relationship allows the image data and the colour measurements to be mapped onto surface models calculated from the range data.

To ensure accuracy of the system, the system may be configured to check its calibration and registration data using control points. To achieve this, control points with a known, planar shape are located within the imaging volume. The system is configured to perform intercomparison between the best fit XYZ control points and those estimated from the bundle adjustment, checking the planarity of the XYZ control points used in the solution to ensure that the calibration is accurate. Furthermore, the system may be configured to analyse image measurement residuals and estimated parameters and compare them to equivalent residuals/parameters taken from historical system usage as well as alternative resection (with the cameras at an alternative angle to the subject) to ensure consistency. Furthermore, the system may be configured to determine the XYZ coordinates of features located within an overlapping area contained in multiple images and compare these to each other to ensure consistency. Furthermore, the position and orientation of each camera may be subsequently determined based on the images and compared to the positions and orientations determined during calibration to ensure consistency.

Whilst the embodiments described herein discuss the quantification of skin diseases, these methods may equally be applied to the quantification of any form of skin disorder or skin condition that is identifiable via discolouration, such as sunburn.

In addition, whilst the embodiments discussed herein discuss the use of range measurements to generate a model of the surface of the skin of a subject, alternative embodiments may generate a model based solely on image data from a network of images made with 2D cameras. This can be achieved by analysing images of the subject taken at a variety of angles in order to generate a surface model of the subject. The colour data from the images may then be mapped onto the surface model to allow any skin abnormalities to be analysed as per steps 108 and 110 of the method of FIG. 2. By utilising only camera data, the system can be implemented more cost effectively. Nevertheless, the use of active range measurements provides a more efficient and effective method for classifying and quantifying skin abnormalities because it is not reliant on finding areas of common detail in each image.

Furthermore, whilst the embodiments described herein utilise range and camera measurements from a fixed position, it is possible to take these measurements from a mobile device, such as a smart phone or tablet computer. This can be combined with the use of continuous measurements (e.g. video) so that the model can be accurately assembled from a large number of measurements taken quickly from various locations around the subject.

In addition, whilst the embodiments described herein utilise colour images, alternative embodiments may classify the skin of a subject based on black and white images. In this case, skin abnormalities may be indicated by a change in shading.

Furthermore, whilst the embodiments described herein may use of a range camera to provide range data indicating the position of the surface of the skin, alternative methods of mapping the surface of the skin may be utilised.

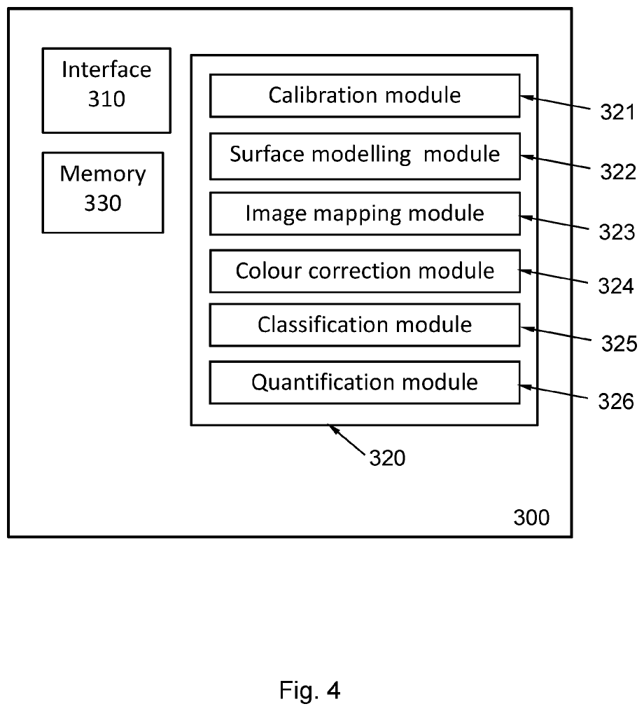

FIG. 4 shows a computing system 300 for determining the nature and/or extent of a skin disorder according to an embodiment. The computing system 300 may be the computing system 20 of FIG. 1. The computing system may implement the methods of FIGS. 3 and 4. The computing system 300 may be any general purpose computing system provided that it is loaded with executable code for performing the methods described herein.

The computing system comprises an interface 310 for receiving one or more images of a subject. The interface may also receive range data of the subject. The computing system 300 further comprises a controller 320 configured to perform the processing steps discussed herein.

The controller 320 can be, by way of example without limitation, a digital processor, a computer microchip, electronic circuitry, or any other form of computing processor.

The computing system 300 also includes memory for storing calibration data to allow the controller to generate a surface model of the skin of the subject and to map the colour data onto the surface model. The memory 330 also stores classification data to allow the controller to classify areas of the textured model into one or more regions affected by a skin condition and one or more regions not affected by the skin condition. The memory 330 is non-volatile memory. The memory 330 stores executable computer-readable instructions which, when executed by the controller 320, cause the controller to implement the methods discussed herein.

The controller 320 further comprises a calibration module 321. The calibration module 321 is configured to calibrate the system based on measurements of known shape and measurements of known colour.

The controller 320 further comprises a surface modelling module 322 configured to determine a surface model of the skin of the subject from measurement data of the location of the skin of the subject. The measurement data may comprise position measurements of the skin, for instance, range data. Alternatively, or in addition, the measurement data may comprise image data. Accordingly, a surface model of the subject may be determined solely from image data, solely from position data, or from a combination of both image and position data.

The controller 320 further comprises an image mapping module 323 configured to map colour data from the images onto the surface model to form an image-mapped surface model of the skin of the subject.

The controller 320 further comprises a colour correction module 324 for correcting the mapped colour data to take account of position, lighting, surface geometry and specularity.

The controller 320 further comprises a classification module 325 for classifying various regions of the image-mapped surface model as either affected by a skin condition or not affected by a skin condition.

The controller 320 further comprises a quantification module 326 for quantifying the nature and/or extent of the skin condition. This may be based on surface area affected by the skin condition and/or the average discolouration of the areas affected by the skin condition.

A system according to the embodiment of FIG. 1 has been tested in 10 patients recruited from the Paediatric Dermatology department in Great Ormond Street Hospital, under standard lighting conditions, in the medical illustration department (professional photographic set up), and data analysed in the Civil, Environmental and Geomatic Engineering (CEGE) department of UCL. This was done under full approval of the London Bloomsbury Research Ethics Committee.

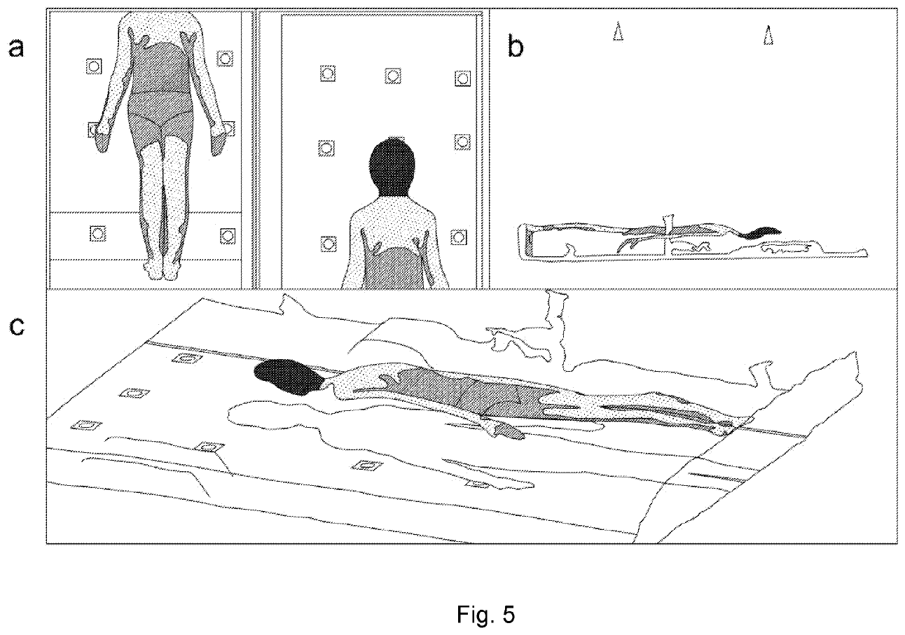

FIG. 5 shows classified images and a classified surface model produced according to an embodiment for a single subject. FIG. 5a shows two camera images of the subject. FIG. 5b shows a 3D reconstruction of the subject based on combined ranging and image information. FIG. 5c shows a shaded view of the 3D reconstruction demonstrating the separation from the reference background. This separation allows the calculation of the surface area affected by the disease as a percentage of the total body surface area. In this case, the percentage affected is 26%. In each of the figures, the areas identified as affected by a skin condition are shown in blue, normal skin is shown in red.

### Advantages

NHS and global health care costs for skin disease are currently rising steeply, with the arrival of biological therapies for common diseases such as psoriasis and recently eczema. The methods presently available are unable to produce the advantages associated with the embodiments described herein.

Firstly, the present methodology is able to provide quantified severity measurements of skin diseases. This is distinguished from imaging systems, such as dermoscopy (hand held magnification aid) or mole mapping (photographic 2D imaging system for monitoring of changes over time) for skin cancer surveillance, which are unable to quantify the extent and severity of multifocal skin diseases being examined.

Secondly, the embodiments descried herein provide a framework for significantly more accurate and objective measurements than existing skin disease severity scoring systems, even when compared to well-validated methods such as the Patient Oriented Eczema Measure (POEM), or the consensus classification system for congenital melanocytic naevi.

Thirdly, the systems and methods described herein are capable of measuring and quantifying a wide variety of skin conditions, including multifocal skin conditions. Alternative methods are unable to provide a holistic quantification of the extent or severity of multifocal skin conditions across the whole body as they are only able to consider isolated regions of the body.

In addition, the systems and methods described herein are able to accurately quantify and track the progression of skin diseases, even in children. Children are difficult to monitor as they are likely to grow between measurements and, in addition, their body proportions change (for example, the head grows much less than the torso—a baby's head is much bigger as a proportion of its length than an adult's—also boys torso's grow longitudinally much more than girls', etc.). This can present challenges when quantifying the severity or extent of a skin condition which is present during childhood.

For instance, current methods extrapolate the size of an affected area of skin up to a Projected Adult Size (an estimated size in adulthood). The differential growth patterns of various body parts makes this particularly difficult when affected areas of skin cross between various body parts (e.g. from the head down to the torso). This can lead to inaccuracies in the assessment of affected surface area.

The embodiments described herein are able to quantify the extent of the skin condition based on the percentage of the overall surface are of the subject that is affected. This therefore accounts for changes in body size so that users can determine whether the skin condition is spreading or receding.

Further embodiments make use of a growth projection mechanism for providing a more accurate projected adult measurement for standardisation of quantification. This makes use of known growth patterns for males and females (including difference in growth speed for different body parts) to transform the model of the subject into a predicted adult model. A projected adult measurement then may be made based on the predicted adult model (e.g. based on the surface area affected in the predicted adult model).

Furthermore, the methods described herein are easy to use and therefore can be applied easily by a wider variety of users. This is in contrast to a number of the current severity measures that require a high level of training to implement.

Narrative market research from members of the British Society of Paediatric Dermatology has demonstrated overwhelming support for an accurate measurement system for skin conditions.

In parallel, patient surveys and patient group engagement prioritised accurate skin measurement system research, and the need for improved personalised treatment based on individual measurements. Two surveys were designed specifically for this project, in collaboration with the PPI co-I on this project. Results were highly supportive of this topic and research questions as Research Priorities. For example, in answer to the question “Do you think it would be useful if there was a camera that could take a picture and measure accurately how extensive and how severe your child's skin disease is?” 95% of respondents ticked yes. In addition, in answer to the question “Do you think developing a new and accurate measurement system should be a priority for birthmark research?” 85% of responses were positive. To a question of prioritising the features of the Skin Sizer system, the desire for accuracy of measurement scored at 90%.

Implementations of the subject matter and the operations described in this specification can be realized in digital electronic circuitry, or in computer software, firmware, or hardware, including the structures disclosed in this specification and their structural equivalents, or in combinations of one or more of them. Implementations of the subject matter described in this specification can be realized using one or more computer programs, i.e., one or more modules of computer program instructions, encoded on computer storage medium for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

While certain arrangements have been described, the arrangements have been presented by way of example only, and are not intended to limit the scope of protection. The inventive concepts described herein may be implemented in a variety of other forms. In addition, various omissions, substitutions and changes to the specific implementations described herein may be made without departing from the scope of protection defined in the following claims.

1. A computer-implemented method for quantifying the nature and/or extent of a skin disorder, the method comprising:
receiving one or more images of skin of a subject;
determining a surface model of the skin of the subject;
mapping the one or more images of the skin of the subject onto the surface model to form an image-mapped surface model of the skin of the subject; and
determining a value for the nature and/or extent of a skin disorder based on the image-mapped surface model of the skin of the subject.

2. The method of claim 1 wherein:
the method further comprises identifying one or more regions of skin that are affected by a skin disorder based on the image-mapped surface model of the skin; and
the value for the nature and/or extent of the skin disorder is determined based on the one or more regions of skin that are affected by the skin disorder.

3. The method of claim 2 further comprising receiving a set of characteristics of skin that is affected by the skin disorder, wherein the identification of the one or more regions of skin that are affected by a skin disorder is based on the set of characteristics.

4. The method of claim 2, wherein the one or more regions of skin that are affected by a skin disorder are identified using a machine learning classifier.

5. The method of claim 2, wherein determining the value for the nature and/or extent of the skin disorder comprises one or more of:
determining the surface area of the one or more regions of skin that are affected by the skin disorder;
determining the proportion of the overall surface area of skin that is affected by the skin disorder;
determining one or more of an average discolouration, an average shading, or an average colouring of the one or more regions of skin that are affected by the skin disorder; and
determining, within the one or more regions of skin that are affected by the skin disorder, the strength of one or more spectral signatures associated one or more corresponding molecules that characterise the skin disorder.

6. The method of claim 1, wherein the method further comprises identifying one or more regions of skin not affected by the skin disorder.

7. The method of claim 6 further comprising receiving a further set of characteristics, the further set of characteristics being of skin that is not affected by the skin disorder, and wherein the identification of the one or more regions of skin not affected by the skin condition is based on the further set of characteristics.

8. The method of claim 1, wherein the method comprises receiving position data indicating the shape of the skin of the patient and wherein the surface model is determined based on the position data.

9. The method of claim 1 wherein the surface model is determined based on the one or more images.

10. A system for quantifying the severity of a skin disorder, the system comprising a processor configured to:
receive one or more images of skin of a subject;
determine a surface model of the skin of the subject;
map the one or more images of the skin of the subject onto the surface model to form an image-mapped surface model of the skin of the subject; and
determine a value for the nature and/or extent of a skin disorder based on the image-mapped surface model of the skin of the subject.

11. A non-transitory computer readable medium comprising computer executable instructions that, when executed by a computer, cause the computer to implement a method comprising:
receiving one or more images of skin of a subject;
determining a surface model of the skin of the subject;
mapping the one or more images of the skin of the subject onto the surface model to form an image-mapped surface model of the skin of the subject; and
determining a value for the nature and/or extent of a skin disorder based on the image-mapped surface model of the skin of the subject.
